(19) United States
(12) Patent Application Publication
(10) Pub. No.: US 2018/0085161 A1
(43) Pub. Date: Mar. 29, 2018

(54) RADIO FREQUENCY ABLATION FOR TREATMENT OF CARDIAC ARRHYTHMIAS

(71) Applicant: Cardiac Pacemakers, Inc, St. Paul, MN (US)

(72) Inventors: Esteban Martin Solorzano Zeledon, Desamparados (Alajuela) (CR); Adrian Valdelomar, La Guacima (Alajuela) (CR)

(21) Appl. No.: 15/712,709

(22) Filed: Sep. 22, 2017

Related U.S. Application Data
(60) Provisional application No. 62/400,540, filed on Sep. 27, 2016.

(51) Int. Cl. A61B 18/14; A61N 1/06

(52) U.S. Cl. CPC A61B 18/1492; A61B 2017/00017; A61N 1/06

(57) ABSTRACT

A catheter includes an elongate catheter body, a catheter tip coupled to a distal end of the elongate catheter body, and a radio-frequency (RF) energy source positioned within the catheter tip. The catheter tip includes a waveguide having a distal end. The RF energy source is spaced from the distal end of the waveguide.

### CROSS-REFERENCE TO RELATED APPLICATION

This application claims priority to Provisional Application No. 62/400,540, filed Sep. 27, 2016, which is herein incorporated by reference in its entirety.

### TECHNICAL FIELD

The present disclosure relates generally to medical devices and, more particularly, to systems, devices and methods related to catheters used to perform ablation functions.

### BACKGROUND

Cardiac ablation is a procedure by which cardiac tissue is treated to inactivate the tissue. The tissue targeted for ablation may be associated with improper electrical activity, for example. Cardiac ablation can lesion the tissue and prevent the tissue from improperly generating or conducting electrical signals.

### SUMMARY

In Example 1, a catheter includes an elongate catheter body, a catheter tip coupled to a distal end of the elongate catheter body, and a radio-frequency (RF) energy source positioned within the catheter tip. The catheter tip includes a waveguide having a distal end. The RF energy source is spaced from the distal end of the waveguide.

In Example 2, the catheter of Example 1, wherein the waveguide is configured and shaped to focus RF energy generated by the RF energy source.

In Example 3, the catheter of any of Examples 1-2, wherein the waveguide includes an opening at the distal end of the waveguide.

In Example 4, the catheter of Example 3, wherein the opening is one of circular shaped and rectangular shaped.

In Example 5, the catheter of any of Examples 3-4, further comprising a cover positioned in the opening.

In Example 6, the catheter of Example 5, wherein the cover comprises a ceramic.

In Example 7, the catheter of any of Examples 5-6, wherein the cover is RF-translucent.

In Example 8, the catheter of any of Examples 5-7, further comprising a temperature sensor at least partially positioned within the cover.

In Example 9, the catheter of any of Examples 3-8, wherein the opening has an area of 0.5-30 mm² at the distal end of the waveguide.

In Example 10, the catheter of any of Examples 1-9, wherein the RF energy source is one of a sphere shape and a rectangle shape.

In Example 11, the catheter of any of Examples 1-10, wherein the RF energy source is spaced from the distal end of the waveguide a distance of 5-12 mm.

In Example 12, the catheter of any of Examples 1-11, wherein the catheter tip is coupled to the elongate catheter body by a weld.

In Example 13, a catheter including an elongate catheter body, a catheter tip coupled to a distal end of the elongate catheter body, and a spiral antenna structure configured to generate radio-frequency (RF) energy. The catheter tip includes an opening at the distal end of the catheter tip. The spiral antenna structure is configured to propagate the generated RF energy through the spiral antenna structure and through the opening.

In Example 14, the catheter of Example 13, further comprising a cover positioned in the opening.

In Example 15, the catheter of Example 14, further comprising a temperature sensor at least partially positioned within the cover.

In Example 16, a catheter includes an elongate catheter body, a catheter tip coupled to a distal end of the elongate catheter body, and a radio-frequency (RF) energy source positioned within the catheter tip. The catheter tip includes a waveguide having a distal end. The RF energy source is spaced from the distal end of the waveguide and at least partially surrounded by the waveguide.

In Example 17, the catheter of Example 16, wherein the waveguide is configured and shaped to focus RF energy generated by the RF energy source.

In Example 18, the catheter of Example 16, wherein the waveguide includes an opening at the distal end of the waveguide.

In Example 19, the catheter of Example 18, wherein the opening is one of circular shaped and rectangular shaped.

In Example 20, the catheter of Example 19, further comprising a cover positioned in the opening.

In Example 21, the catheter of Example 20, wherein the cover comprises a ceramic.

In Example 22, the catheter of Example 20, wherein the cover is RF-translucent.

In Example 23, the catheter of Example 20, further comprising a temperature sensor at least partially positioned within the cover.

In Example 24, the catheter of Example 19, wherein the opening is positioned at a distal end of the catheter tip.

In Example 25, the catheter of Example 19, wherein the opening is positioned between a distal end and proximal end of the catheter tip.

In Example 26, the catheter of Example 16, wherein the RF energy source is one of a sphere shape and a rectangle shape.

In Example 27, the catheter of Example 16, wherein the RF energy source is spaced from the distal end of the waveguide a distance of 5-12 mm.

In Example 28, the catheter of Example 16, wherein a diameter of the waveguide is greater than a diameter of the elongate catheter body.

In Example 29, the catheter of Example 16, wherein the waveguide is configured to direct RF energy generated by the RF energy source in a direction parallel to an axial direction of the catheter tip.

In Example 30, the catheter of any of Examples 1-29, further comprising an actuator coupled to the RF energy source and configured to position the RF energy source within the catheter tip.

In Example 31, the catheter of Example 16, wherein the waveguide includes an inner wall that is curved.

In Example 32, the catheter of Example 16, wherein the waveguide includes inner walls that taper as the inner walls extend in a direction towards the distal end of the waveguide.

In Example 33, a catheter includes an elongate catheter body, a catheter tip coupled to a distal end of the elongate catheter body, and a spiral antenna structure configured to generate radio-frequency (RF) energy. The catheter tip includes an opening at the distal end of the catheter tip. The spiral antenna structure is configured to propagate the generated RF energy through the spiral antenna structure and through the opening.

In Example 34, the catheter of Example 33, wherein the opening is one of circular shaped and rectangular shaped.

In Example 35, a catheter includes means for generating a radio-frequency (RF) energy field and a catheter tip including means for focusing the RF energy field.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 shows a catheter system.

FIG. 2 shows a schematic side view of a portion of a catheter, in accordance with certain embodiments of the present disclosure.

FIG. 3 shows a schematic, exploded, perspective view of a portion of a catheter, in accordance with certain embodiments of the present disclosure.

FIG. 4 shows a schematic, exploded, perspective view of a portion of a catheter, in accordance with certain embodiments of the present disclosure.

FIG. 5 shows a schematic, exploded, perspective view of a portion of a catheter, in accordance with certain embodiments of the present disclosure.

FIG. 6 shows a schematic, exploded, perspective view of a portion of a catheter, in accordance with certain embodiments of the present disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the invention as defined by the appended claims.

### DETAILED DESCRIPTION

Various cardiac abnormalities can be attributed to improper electrical activity of cardiac tissue. Such improper electrical activity can include, but is not limited to, generation of electrical signals, conduction of electrical signals, and/or compression of the tissue in a manner that does not support efficient and/or effective cardiac function. For example, an area of cardiac tissue may become electrically active prematurely or otherwise out of synchrony during the cardiac cycle, causing the cardiac cells of the area and/or adjacent areas to contract out of rhythm. The result is an abnormal cardiac contraction that is not timed for optimal cardiac output. In some cases, an area of cardiac tissue may provide a faulty electrical pathway (e.g., a short circuit) that causes an arrhythmia, such as atrial fibrillation or supraventricular tachycardia. In some cases, inactive tissue (e.g., scar tissue) may be preferable to malfunctioning cardiac tissue.

Cardiac ablation is a procedure by which cardiac tissue is treated to inactivate the tissue. The tissue targeted for ablation may be associated with improper electrical activity, as described above. Cardiac ablation can create lesions in the tissue and prevent the tissue from improperly generating or conducting electrical signals. For example, a line, a circle, or other formation of ablated cardiac tissue can block the propagation of errant electrical signals. In some cases, cardiac ablation is intended to cause the death of cardiac tissue and to have scar tissue reform over the lesion, where the scar tissue is not associated with the improper electrical activity. Ablation therapies include radiofrequency ablation, cyroablation, microwave ablation, laser ablation, and surgical ablation, among others.

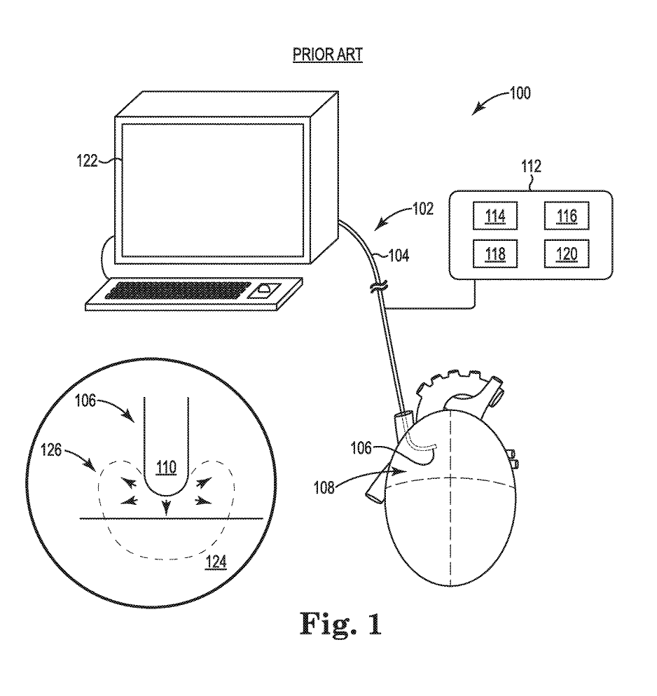

FIG. 1 shows a catheter system 100 including a catheter 102 comprising an elongated catheter body 104 and a catheter tip 106, which is configured to be positioned within a heart 108. The catheter 102 includes an ablation electrode 110 coupled to the catheter tip 106. The system 100 also includes control circuitry 112 (including a memory 114, processor 116, energy generator 118, and display controller 120) and a display 122 for carrying out various functions of the catheter system 100.

In operation, the ablation electrode 110 contacts targeted cardiac tissue to deliver ablative energy to the cardiac tissue, thus ablating the tissue to form a lesion, which can treat cardiac rhythm disturbances or abnormalities. The ablation electrode 110 in FIG. 1 is a radio frequency (RF) ablation electrode, which delivers RF energy to cardiac tissue 124. A field of the RF energy generated by the ablation electrode 110 is shown in dotted lines as reference number 126. The shape of the field 126 is due to the shape of the catheter tip 106, which behaves like a monopole antenna that propagates RF energy in all directions. In such a configuration, RF energy is directed not only to the targeted cardiac tissue but also to blood and untargeted tissue proximate the ablation electrode 110. Features of the present disclosure are accordingly directed to catheter tip designs with targeted RF energy propagation to enhance the efficiency of the RF energy transfer to the target tissue, and to provide increased predictability in lesion formation.

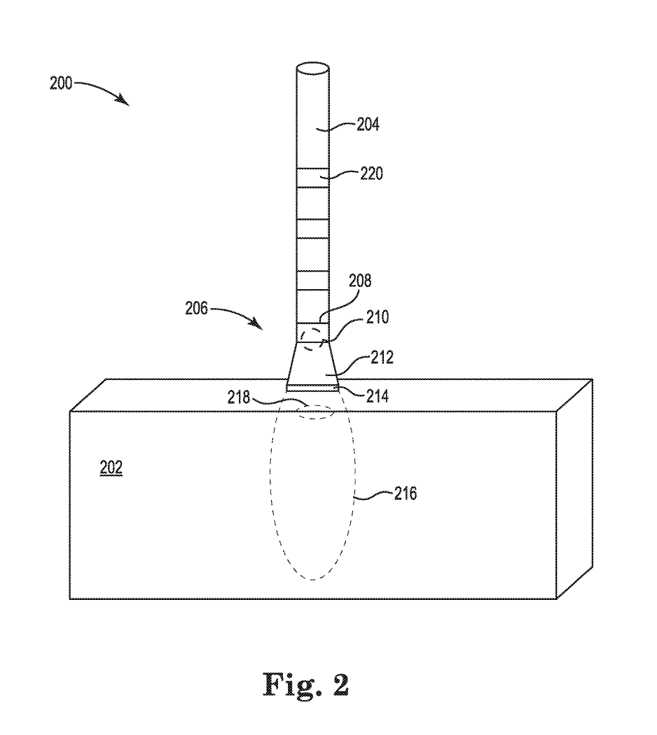

FIG. 2 shows a schematic side view of a portion of a catheter 200 positioned adjacent tissue 202. The catheter 200 can be utilized in a system like the catheter system 100 shown in FIG. 1. The catheter 200 includes an elongate catheter body 204 and a catheter tip 206. The catheter tip 206 is coupled to a distal end 208 of the catheter body 204, for example, by a weld (e.g., solder) or an adhesive. The catheter 200 includes an energy source 210 positioned within the catheter tip 206 that emits energy (e.g., radio frequency energy) towards a waveguide 212 and tissue 202. The energy source 210 is electrically coupled to an energy generator (e.g., energy generator 118 shown in FIG. 1). In some embodiments, the energy source 210 can be considered to be an antenna-like point source in that energy is emitted (or broadcast) in all directions. The energy source 210 can comprise metals such as stainless steel, titanium, gold, and other biocompatible metal alloys. In some embodiments, the energy source 210 is centrally positioned within the catheter tip 206. In some embodiments, the energy source 210 is electrically isolated from the catheter tip 206. For example, the energy source 210 can be coupled to the catheter tip 206 by an electrically-insulating material (e.g., ceramics, nonconductive biocompatible materials) surrounding the energy source 210. Although only a single energy source 210 is shown in FIG. 2, the catheter 200 can include multiple energy sources, which, for example, can generate energy at different frequencies. In some embodiments, a single energy source is used to generate energy at several different and/or modulated frequencies.

The waveguide 212 is configured and shaped to focus energy generated by the energy source 210, which is spaced from a distal end 214 of the waveguide 212. Features of the waveguide 212 (e.g., wall shape, material) shape an energy field generated by the energy source 210 such that the energy field can be used to create lesions with better controlled dimensions (e.g., area, depth, shape) and such that less energy is directed to surrounding blood and untargeted tissue. For example, the waveguide 212 can collimate the energy field generated by the energy source 210 such that signals within the energy field are in a direction substantially perpendicular to tissue 202. Reference number 216 in FIG. 2 represents an energy field propagated by the catheter tip 206 towards a target section 218 of tissue 202. Compared to the energy field 126 of FIG. 1, it can be seen that a larger percentage of energy field 216 of FIG. 2 is directed towards tissue 202 as opposed to being directed to surrounding blood and untargeted tissue.

Example materials for the waveguide 212 include materials comprising biocompatible metal alloys such as platinum, iridium, stainless steel, titanium alloys, gold, tantalum, and various combinations of such metal alloys (e.g., Pt₉₀Ir₁₀). Other materials for the waveguide 212 include composite materials such as flexible circuit films (e.g., polyimide) and/or composite materials having an organic-based substrate with layers of biocompatible metal alloys such as those mentioned above. Example shapes for the waveguide 212—described in more detail below—include shapes such as horn shapes with various cross sections, including circular and rectangular cross sections. Although the waveguide 212 is shown in FIG. 2 as directing energy in an axial direction, embodiments described below can feature waveguides that direct energy in a radial direction. Further, although the waveguide 212 is shown as having a diameter larger than a diameter of the catheter body 204, embodiments described below can feature waveguides that are shaped to have the same or a smaller diameter than a diameter of a catheter body.

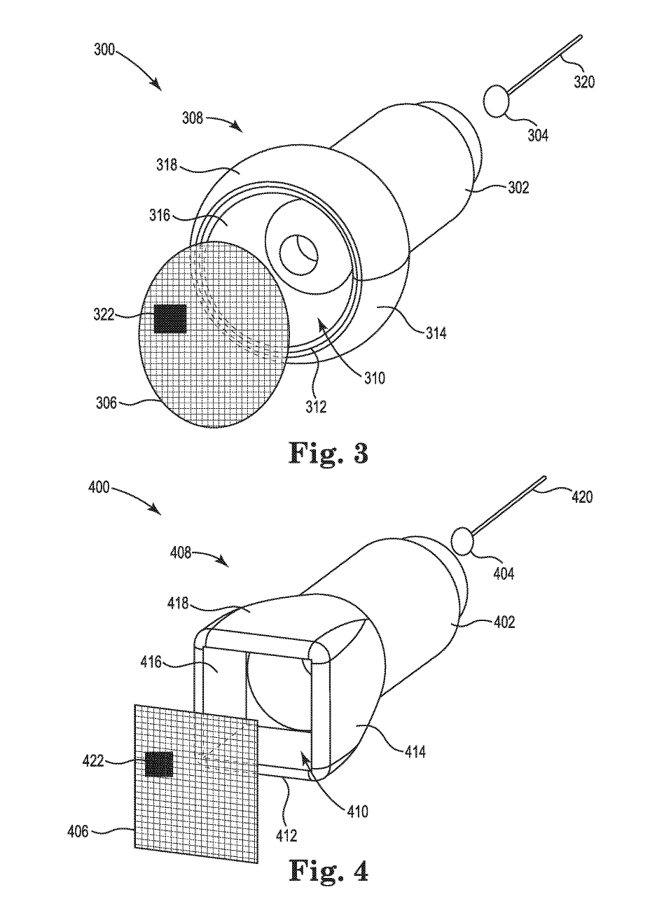

In contrast to the catheter 100 in FIG. 1, the catheter 200 of FIG. 2 (and the catheters shown in FIGS. 3-6) and its energy source 210 and catheter tip 206 with waveguide 212 can provide ablative energy to tissue without tissue being in direct contact with the energy source 210 and/or catheter tip 206. For example, for the catheter 100 in FIG. 1 to ablate tissue, the ablation electrode 110 must be energized and directly contact the tissue. In some embodiments, contactless ablation can occur when the energy source 210 operates at a high frequency. In some embodiments, the catheter 200 provides direct-contact ablative energy when energy source 210 operates at a high frequency. At higher frequencies (on the order of MHz), contactless current (e.g., high frequencies)—sometimes referred to as displacement current—progressively dominates over conduction current (e.g., low frequencies). The energy source 210 can also operate at a combination of low and high frequency to provide both contact and contactless energy delivery capability. The catheter 200 can include additional features such as mapping electrodes 220, various sensors, various actuators, etc., and can be irrigated or non-irrigated.

FIG. 3 shows a schematic, exploded, perspective view of a portion of a catheter 300. The catheter 300 includes a catheter tip 302, an energy source 304, and a cover 306. The catheter tip 302 includes a waveguide portion 308 having a circular-shaped opening 310 at a distal end 312 of the waveguide portion 308. In some embodiments, the opening 310 has an area of 0.5-30 mm² at the distal end 312 of the waveguide portion 308. The waveguide portion 308 includes an outer wall 314 and an inner wall 316 and a waveguide body 318 there in between.

When the catheter 300 is assembled, the energy source 304 is positioned within the catheter tip 302 spaced from the distal end 312 of the waveguide portion 308 at a proximal end of the waveguide portion 308. In some embodiments, the energy source 304 is spaced from the distal end 312 of the waveguide portion 308 by a distance of 5-12 mm. In some embodiments, the energy source 304 is attached to an actuator (e.g., plunger, rod) that can move the energy source 304 to change the distance between the energy source 304 and the distal end 312 of the waveguide portion 308. The actuator can be coupled to a deployment mechanism that is controllable at a handle of the catheter 300. The energy source 304 can be secured within the catheter tip 302 by a ceramic or other nonconductive biocompatible material to electrically isolate the energy source 304 from catheter tip 302, etc. The energy source 304 is electrically coupled to an energy generator (e.g., energy generator 118 shown in FIG. 1) via connection 320, which can be a wire or cable that transmits an electrical signal. The connection 320 can extend through a lumen within the catheter 300 between the energy source 304 and the energy generator. Although the energy source 304 is shown as being sphere-shaped in FIG. 3, the energy source 304 can be other shapes. The energy source 304 can comprise metals such as stainless steel, titanium, gold, and other biocompatible metal alloys.

During use, the energy source 304 generates and directs an energy field (e.g., radio-frequency energy) towards the waveguide portion 308 and tissue. The waveguide portion 308 shapes the generated energy field to control parameters of tissue ablation. For example, because the waveguide portion 308 shown in FIG. 3 has a circular opening 310, the energy field (and therefore a resulting lesion) would form a similar, circular shape. In particular, the inner wall 316 of the waveguide portion 308 helps provide the resulting shape of the energy field. The inner wall 316 directs energy that would otherwise propagate towards blood or untargeted tissue towards the opening 310 and targeted tissue. In some embodiments, the inner wall 316 includes a sloped portion that collimates the energy field towards the opening 310 and target tissue. The waveguide body 318 is sufficiently thick such that few to no signals of the energy field are transmitted through the waveguide body 318.

When the catheter 300 is assembled, the cover 306 is positioned, at least partially, within the opening 310 such that an internal cavity is formed within the catheter tip 302 between the cover 306 and inner wall 316 of the waveguide portion 308. The cover 306 prevents blood from entering and building up in the internal cavity. In some embodiments, the internal cavity is filled with radio-frequency transparent material such as ceramics or biocompatible plastics that are radio-frequency transparent. In some embodiments, the internal cavity is hermetically isolated with a lid (e.g., cover 306) made of radio-frequency friendly ceramic or biocompatible plastic of sufficient thickness to prevent fluid ingress. One or more sensors 322 can be embedded, at least partially, within the cover 306. For example, the sensor 322 can be a temperature sensor (e.g., thermocouple, thermistor) that is partially embedded in the cover 306 such that the sensor 322 senses temperature of blood and/or tissue during an ablation procedure. The sensor 322 may be partially exposed (e.g., not fully encapsulated by the cover 306) so that the sensor 322 can accurately sense temperature. The cover 306 can comprise a biocompatible, nonconductive material, such as a ceramic, to thermally isolate the sensor 322 from other components of the catheter 300. The material of the cover 306 also permits energy, such as radio-frequency energy, generated by the energy source 304 to be transmitted through the cover 306 and towards tissue.

FIG. 4 shows a schematic, exploded, perspective view of a portion of a catheter 400. The catheter 400 includes a catheter tip 402, an energy source 404, and a cover 406. The catheter tip 402 includes a waveguide portion 408 having a rectangular-shaped opening 410 at a distal end 412 of the waveguide portion 408. In some embodiments, the opening 410 has an area of 0.5-30 mm² at the distal end 412 of the waveguide portion 408. The waveguide portion 408 includes an outer wall 414, an inner wall 416, and a waveguide body 418 there in between. Although FIG. 4 shows the inner wall 416 having four sections that are flat and parallel to a longitudinal axis of the catheter 400, the sections of the inner wall 416 can be tapered, curved, etc. Further, the inner wall 416 can include fewer or more sections than shown in FIG. 4.

When the catheter 400 is assembled, the energy source 404 is positioned within the catheter tip 402 spaced from the distal end 412 of the waveguide portion 408 at a proximal end of the waveguide portion 408. In some embodiments, the energy source 404 is spaced from the distal end 412 of the waveguide portion 408 by a distance of 5-12 mm. In some embodiments, the energy source 404 is attached to an actuator (e.g., plunger, rod) that can change the distance between the energy source 404 and the distal end 412 of the waveguide portion 408. The actuator can be coupled to deployment mechanism that is controllable at a handle of the catheter 400. The energy source 404 can be secured within the catheter tip 402 by a ceramic or other nonconductive biocompatible material to electrically isolate the energy source 404 from catheter tip 402, etc. The energy source 404 is electrically coupled to an energy generator (e.g., energy generator 118 shown in FIG. 1) via connection 420, which can be a wire that transmits an electrical signal. The connection 420 can extend through a lumen within the catheter 400 between the energy source 404 and the energy generator. Although the energy source 404 is shown as being sphere-shaped in FIG. 4, the energy source 404 can be other shapes.

During use, the energy source 404 generates and directs an energy field (e.g., radio-frequency energy) towards the waveguide portion 408 and tissue. The waveguide portion 408 shapes the energy field to control parameters of tissue ablation. For example, because the waveguide portion 408 shown in FIG. 4 has a rectangular opening 410, the energy field (and therefore a resulting lesion) would form a similar, rectangular shape. In particular, the sections of the inner wall 416 of the waveguide portion 408 help provide the resulting shape of the energy field. The inner wall 416 directs energy that would otherwise propagate towards blood or untargeted tissue towards the opening 410 and targeted tissue. The waveguide body 418 is sufficiently thick such that few to no signals of the energy field are transmitted through the waveguide body 418.

When the catheter 400 is assembled, the cover 406 is positioned, at least partially, within the opening 410 such that an internal cavity is formed within the catheter tip 402 between the cover 406 and inner wall 416 of the waveguide portion 408. The cover 406 prevents blood from entering and building up in the internal cavity. In some embodiments, the internal cavity is filled with radio-frequency transparent material such as ceramics or biocompatible plastics that are radio-frequency transparent. In some embodiments, the internal cavity is hermetically isolated with a lid (e.g., cover 406) made of radio-frequency friendly ceramic or biocompatible plastic of sufficient thickness to prevent fluid ingress. One or more sensors 422 and/or actuators can be embedded, at least partially, within the cover 406. For example, the sensor 424 can be a temperature sensor (e.g., thermocouple, thermistor) that is partially embedded in the cover 406 such that the sensor 422 senses temperature of blood and/or tissue during an ablation procedure. The sensor 422 may be partially exposed (e.g., not fully encapsulated by the cover 406) so that the sensor 422 can accurately sense temperature. The cover 406 can comprise a biocompatible, nonconductive material, such as a ceramic, to thermally isolate the sensor 422 from other components of the catheter 400. The material of the cover 406 also permits energy, such as radio-frequency energy, generated by the energy source 404 to be transmitted through the cover 406 and towards tissue.

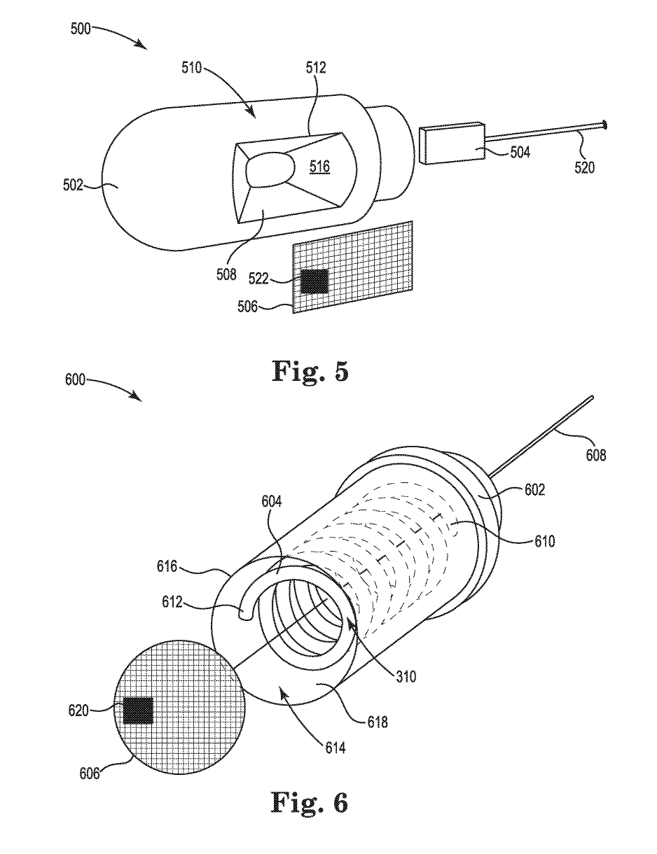

FIG. 5 shows a schematic, exploded, perspective view of a portion of a catheter 500. The catheter 500 includes a catheter tip 502, an energy source 504, and a cover 506. The catheter tip 502 includes a waveguide portion 508 having a rectangular-shaped opening 510 at a distal end 512 of the waveguide portion 508. In some embodiments, the opening 510 has an area of 0.5-30 mm² at the distal end 512 of the waveguide portion 508. The waveguide portion 508 includes sections of an inner wall 516 that extend and taper from an inner wall or a lumen within the catheter tip 502 to an exterior surface 514 of the catheter tip 502.

When the catheter 500 is assembled, the energy source 504 is positioned within the catheter tip 502 spaced from the distal end 512 of the waveguide portion 508. In some embodiments, the energy source 504 is spaced from the distal end 512 of the waveguide portion 508 by a distance of 0.5-2 mm. In some embodiments, the energy source 504 is attached to an actuator (e.g., plunger, rod) that can change the distance between the energy source 504 and the distal end 512 of the waveguide portion 508. The actuator can be coupled to deployment mechanism that is controllable at a handle of the catheter 500. The energy source 504 can be secured within the catheter tip 502 by a ceramic or other nonconductive biocompatible material to electrically isolate the energy source 504 from catheter tip 502, etc. The energy source 504 is electrically coupled to an energy generator (e.g., energy generator 118 shown in FIG. 1) via connection 520, which can be a wire that transmits an electrical signal. The connection 520 can extend through a lumen within the catheter 500 between the energy source 504 and the energy generator. In FIG. 5, the energy source 504 is shown as being a rectangular tablet shape although other shapes can be used.

During use, the energy source 504 generates and directs an energy field (e.g., radio-frequency energy) towards the waveguide portion 508 and tissue. The waveguide portion 508 shapes the energy field to control parameters of tissue ablation. For example, because the waveguide portion 508 shown in FIG. 5 has a rectangular opening 510, the energy field (and therefore a resulting lesion) would form a similar, rectangular shape. In particular, the sections of the inner wall 516 of the waveguide portion 508 help provide the resulting shape of the energy field. The inner wall 516 directs energy that would otherwise propagate towards blood or untargeted tissue towards the opening 510 and targeted tissue.

When the catheter 500 is assembled, the cover 506 is positioned, at least partially, within the opening 510 such that an internal cavity is formed within the catheter tip 502 between the cover 506 and inner wall 516 of the waveguide portion 508. The cover 506 prevents blood from entering and building up in the internal cavity. In some embodiments, the internal cavity is filled with radio-frequency transparent material such as ceramics or biocompatible plastics that are radio-frequency transparent. In some embodiments, the internal cavity is hermetically isolated with a lid (e.g., cover 506) made of radio-frequency friendly ceramic or biocompatible plastic of sufficient thickness to prevent fluid ingress. One or more sensors 522 and/or actuators can be embedded, at least partially, within the cover 506. For example, the sensor 522 can be a temperature sensor (e.g., thermocouple, thermistor) that is partially embedded in the cover 506 such that the sensor 522 senses temperature of blood and/or tissue during an ablation procedure. The sensor 522 may be partially exposed (e.g., not fully encapsulated by the cover 506) so that the sensor 522 can accurately sense temperature. The cover 506 can comprise a biocompatible, nonconductive material, such as a ceramic, to thermally isolate the sensor 522 from other components of the catheter 500. The material of the cover 506 also permits energy, such as radio-frequency energy, generated by the energy source 504 to be transmitted through the cover 506 and towards tissue.

FIG. 6 shows a schematic, exploded, perspective view of a portion of a catheter 600. The catheter 600 includes a catheter tip 602, a spiral antenna structure 604, and a cover 606.

The spiral antenna structure 604 is electrically coupled to an energy generator (e.g., energy generator 118 shown in FIG. 1) via connection 608, which can be a wire or cable that transmits an electrical signal. The connection 608 can extend through a lumen within the catheter 600 between the spiral antenna structure 604 and the energy generator. The spiral antenna structure 604 functions as both an energy source and waveguide. The spiral antenna structure 604 receives the electrical signal transmitted from the energy generator at a proximal end 610 of the spiral antenna structure 604, generates energy (e.g., radio-frequency energy) in response to the electrical signal, and propagates the generated energy through the spiral antenna structure 604 towards a distal end 612 of the spiral antenna structure 604. The energy generated by and propagated through the spiral antenna structure 604 is directed towards an opening 614 at a distal end 616 of the catheter tip 602 towards tissue.

When the catheter 600 is assembled, the cover 606 is positioned, at least partially, within the opening 614 such that an internal cavity is formed within the catheter tip 602 between the cover 606 and an inner wall 618 of the catheter tip 602. In some embodiments, the opening 614 has an area of 0.5-30 mm². The cover 606 prevents blood from entering and building up in the internal cavity. In some embodiments, the internal cavity is filled with radio-frequency transparent material such as ceramics or biocompatible plastics that are radio-frequency transparent. In some embodiments, the internal cavity is hermetically isolated with a lid (e.g., cover 606) made of radio-frequency friendly ceramic or biocompatible plastic of sufficient thickness to prevent fluid ingress. One or more sensors 620 and/or actuators can be embedded, at least partially, within the cover 606. For example, the sensor 620 can be a temperature sensor (e.g., thermocouple, thermistor) that is partially embedded in the cover 606 such that the sensor 620 senses temperature of blood and/or tissue during an ablation procedure. The sensor 620 may be partially exposed (e.g., not fully encapsulated by the cover 606) so that the sensor 620 can accurately sense temperature. The cover 606 can comprise a biocompatible, nonconductive material, such as a ceramic, to thermally isolate the sensor 620 from other components of the catheter 600. The material of the cover 606 also permits energy, such as radio-frequency energy to be transmitted through the cover 606 and towards tissue.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A catheter comprising:
an elongate catheter body;
a catheter tip coupled to a distal end of the elongate catheter body, wherein the catheter tip includes a waveguide having a distal end; and
a radio-frequency (RF) energy source positioned within the catheter tip and spaced from the distal end of the waveguide, wherein the RF energy source is at least partially surrounded by the waveguide.

2. The catheter of claim 1, wherein the waveguide is configured and shaped to focus RF energy generated by the RF energy source.

3. The catheter of claim 1, wherein the waveguide includes an opening at the distal end of the waveguide.

4. The catheter of claim 3, wherein the opening is one of circular shaped and rectangular shaped.

5. The catheter of claim 4, further comprising:
a cover positioned in the opening.

6. The catheter of claim 5, wherein the cover comprises a ceramic.

7. The catheter of claim 5, wherein the cover is RF-translucent.

8. The catheter of claim 5, further comprising:
a temperature sensor at least partially positioned within the cover.

9. The catheter of claim 4, wherein the opening is positioned at a distal end of the catheter tip.

10. The catheter of claim 4, wherein the opening is positioned between a distal end and proximal end of the catheter tip.

11. The catheter of claim 1, wherein the RF energy source is one of a sphere shape and a rectangle shape.

12. The catheter of claim 1, wherein the RF energy source is spaced from the distal end of the waveguide a distance of 5-12 mm.

13. The catheter of claim 1, wherein a diameter of the waveguide is greater than a diameter of the elongate catheter body.

14. The catheter of claim 1, wherein the waveguide is configured to direct RF energy generated by the RF energy source in a direction parallel to an axial direction of the catheter tip.

15. The catheter of claim 1, further comprising:
an actuator coupled to the RF energy source and configured to position the RF energy source within the catheter tip.

16. The catheter of claim 1, wherein the waveguide includes an inner wall that is curved.

17. The catheter of claim 1, wherein the waveguide includes inner walls that taper as the inner walls extend in a direction towards the distal end of the waveguide.

18. A catheter comprising:
an elongate catheter body;
a catheter tip coupled to a distal end of the elongate catheter body, the catheter tip including an opening at the distal end of the catheter tip; and
a spiral antenna structure configured to generate radio-frequency (RF) energy and propagate the generated RF energy through the spiral antenna structure and through the opening.

19. The catheter of claim 18, wherein the opening is one of circular shaped and rectangular shaped.

20. A catheter comprising:
means for generating a radio-frequency (RF) energy field; and
a catheter tip including means for focusing the RF energy field.
